(19) United States
(12) Patent Application Publication
(10) Pub. No.: US 2026/0012028 A1
(43) Pub. Date: Jan. 8, 2026

(54) PROTECTIVE CHARGING CASE

(71) Applicant: Byteexplain Limited, Dublin (IE)

(72) Inventor: John A. Cogan, Dublin (IE)

(21) Appl. No.: 19/009,827

(22) Filed: Jan. 3, 2025

Related U.S. Application Data
(63) Continuation of application No. PCT/EP2023/068626, filed on Jul. 5, 2023.

(30) Foreign Application Priority Data
Jul. 5, 2022 (GB) 2209890.9

(51) Int. Cl. H02J 7/00; A61B 5/00; H02J 50/10

(52) U.S. Cl. CPC H02J 7/0044; H02J 50/10; A61B 5/4557; A61B 2560/0214; H02J 7/0047

(57) ABSTRACT

A charging case for an intraoral device includes a storage compartment surrounded by a peripheral wall extending between a top and a base. A charging output supported by the peripheral wall faces into the storage compartment to transfer energy to the intraoral device contactlessly by induction. Internal locating formations form a pocket for receiving and engaging a protruding sensor part of the intraoral device, holding a receiving input of the intraoral device in close proximity and alignment with the charging output when the case is closed.

### CROSS-REFERENCE TO RELATED APPLICATIONS

This application is a continuation application of and claims priority to International Patent Application No. PCT/EP2023/068626 filed Jul. 5, 2023, which also claims priority to Great Britain Patent Application GB 2209890.9 filed Jul. 5, 2022, the contents of each of which is hereby incorporated by reference in its entirety.

### INTRODUCTION

This invention relates to a protective case that is capable of providing or conveying electric charge to an intraoral device housed temporarily within the case.

### BACKGROUND

Smart wearable devices have become commonplace in society and come in various forms with a wide spectrum of functionality. The uptake of smart wearable devices has led to a requirement for ancillary devices such as protective cases and chargers to support their use.

The invention is particularly concerned with intraoral devices that are configured to be placed in the mouth or buccal cavity. Such devices can be used to monitor mandibular activity, for example to monitor bruxing during sleep, as well as to measure other biometrics. They can come in various forms, for example being encapsulated in or attached to a retainer or mouthguard.

Protective cases are well known to protect intraoral devices such as mouthguards and splints from damage and from becoming soiled or contaminated when they are not in use. US 2004/0244805 discloses an example of a mouthguard storage case. Such cases are typically moulded from a rigid polymer that can be cleaned to mitigate bacterial growth within the case. Effective cleaning or sterilisation may require the entire case to be immersed in water or a cleaning solution.

Contactless inductive charging of encapsulated devices is also well known. For example, Qi is an open interface standard that defines wireless power transfer using inductive charging over distances of up to four centimetres. Inductive charging in this way allows the electronics of a device to remain hermetically sealed, minimising any risk of contamination from within the device or ingress of water or other fluids that could damage the device.

Inductive charging relies upon proximity and alignment between transmitting and receiving coils or loops, the former being implemented in a charging unit and the latter being implemented in the device to be charged. Typically, a charging period takes a matter of hours to complete and therefore the device will usually be left unattended while charging.

Failing to ensure and maintain the necessary proximity and alignment for the full charging period may result in the device being charged inadequately or not at all. Frustratingly, the user may then find that the device is unavailable for use when it is needed or must wait for a long time until the device has been charged afresh.

The challenge of proximity and alignment between transmitting and receiving loops is compounded because mouthguards or other intraoral devices are often bespoke in shape and size, being tailored to the contours of the user's mouth. Their complex, variable shape does not lend itself to effective induction coupling with a universal charger and especially not with commonly available charging mats or pads that are designed to charge a device such as a mobile phone that is laid flat on top of them.

US 2019/110746 shows an example of a case capable of charging an intraoral device held within it. The case described uses a bespoke reproduction of the user's dentition to hold the device in alignment with the coils to allow charge transfer to occur. A bespoke solution such as this inhibits cost effective mass production.

Other examples of cases for intraoral devices featuring incorporated electronics with varying levels of functionality can be found in US 2020/147473, US 2012/172677, US 2016/100924, and GB 2595723.

Where a device is intended to be used in the mouth, it is desirable not to leave the device unprotected during a charging period. Consequently, it is desirable to charge the device when the device is in a protective case. However, a case can block effective inductive charging by increasing the distance between the transmitting and receiving coils and by interposing a solid barrier between the coils.

A case also forms a visual barrier because it can prevent a user seeing status indicators on a device within, such as a light to confirm whether or not the device is charging or that charging is complete. Consequently, there is a risk that when placed in a case, the device will not charge and that a user will not notice the failure to charge until the device is removed again from the case, possibly several hours later.

In principle, a case equipped with a transmitting coil could serve as an induction charger for an intraoral device housed within. However, such an arrangement would also face problems. For example, as noted above, such a case may need to be immersed in liquid for cleaning from time to time. In this respect, a charger usually has a wired power connection and this would present challenges of sealing, especially if the case requires a socket to receive a plug-in power cable. Also, the complex, variable and bespoke shape of an intraoral device continues to present challenges of proximity and alignment between transmitting and receiving loops even if the device is charging within a case.

It is against this background that the present invention has been devised.

### SUMMARY

The invention resides in a charging case for an intraoral device, the case comprising: a storage compartment surrounded by a peripheral wall extending between a top and a base; and a charging output supported by the peripheral wall and facing into the storage compartment.

The peripheral wall may comprise a generally U-shaped arcuate portion. In that example, the charging output may be offset to a side of the arcuate portion and may be adjacent to an end of a limb of the U-shape. The arcuate portion of the peripheral wall may be generally U-shaped internally and externally.

The peripheral wall may be thickened to define a chamber for electronics housed within the peripheral wall. That chamber is preferably sealed. The charging output may be housed within a recess in the peripheral wall and that recess may conveniently be formed in the thickened portion of the peripheral wall. Preferably, a wall of the recess corresponding to the charging output is substantially planar.

The peripheral wall may be substantially orthogonal to a substantially planar base of the charging case and may be integral with and upstanding from the base. This defines an upwardly open receptacle that can be closed by a moveable or removable top to define the storage compartment.

The charging output may be contactless, for example comprising a generally planar induction loop located in or near the peripheral wall and may also comprise a magnetic coupling. Similarly, the case may further comprise a charging input that, may be contactless and may further comprise a magnetic coupling. A contactless charging input may comprise a generally planar induction loop located on or in the base, which may be in substantially parallel planes. An induction loop of the charging input may be larger in diameter than an induction loop of the charging output. The charging input and the charging output could lie or act in mutually transverse or orthogonal planes or directions.

The case may further comprise an onboard energy store that is capable of charging the intraoral device when the case is not connected to an external source of electrical power.

A charging system, comprising the charging input and the charging output, could be contained within a charging system module that may be detachable from a body of the case. The charging system module may also include the onboard energy store. The module may be substantially ‘L’ shaped with an elongate base and comparatively short upright. The module and the body of the case could feature complementary rail formations that may be substantially parallel to the base of the case.

The top or the peripheral wall of the case may be at least partially transparent or translucent.

The case may comprise at least one status indicator. The or each status indicator may be disposed on or in the top or the peripheral wall.

The top of the case may comprise at least one locating formation that extends into the storage compartment when the case is closed. Where the case has a recess in the peripheral wall, the locating formation may align with and be opposed to the recess when the case is closed. The locating formation may be spaced from the outer wall of the recess to define a pocket between the locating formation and the outer wall when the case is closed.

The inventive concept also embraces a combination of the charging case of the invention and an intraoral device that is arranged to fit into the storage compartment and to receive energy from the charging output.

Elegantly, the intraoral device may be substantially constrained in all degrees of freedom by formations within the case, which formations may include the abovementioned recess. The internal formations of the case may form at least one pocket for receiving and engaging a protruding part of the intraoral device, such as a sensor module, when the case is closed.

The charging output of the case is suitably aligned with and proximate to a receiving input of the intraoral device when the intraoral device is disposed in the storage compartment. In this instance, the charging output of the case may comprise an induction loop that substantially corresponds in size to an induction loop of the receiving input of the intraoral device.

The intraoral device may require elastic deformation to fit within the storage compartment. That deformation may involve inward displacement of opposed limbs of a U-shaped part of the intraoral device. Advantageously, the elastic reaction of the intraoral device can help to locate the device within the storage compartment of the case and to maintain effective coupling between the intraoral device and the charging output of the case.

The inventive concept extends to a corresponding method of charging an intraoral device. The method comprises: coupling a receiving input of the intraoral device to a charging output of a protective charging case; coupling a charging input of the case to an external power supply; and charging the intraoral device with energy provided from the power supply via the case. The couplings may be effected contactlessly.

The method may also comprise: providing energy from the power supply to the case; storing the energy in the case; and subsequently transferring the stored energy from the case to the intraoral device. Energy may be provided from the power supply to the case before coupling the receiving input of the intraoral device to the charging output of the case. The stored energy may be transferred from the case to the intraoral device when the external power supply is no longer coupled to the charging input of the case.

When the intraoral device has been charged, the method may further comprise: removing a charging system module from a body of the case, that module containing the charging output and the charging input of the case; and cleaning the body of the case.

Thus, the invention resides in a case for simultaneously protecting, and providing or conveying electrical charge from an external power source to, a smart electronic intraoral device contained within. In preferred embodiments, apparatus of the invention comprises a charging case for temporarily housing the intraoral device while is it not in use, a means of holding the intraoral device in place within the case and a charging means to receive electrical charge from an external power source and to transmit that electrical charge to the device.

The invention arises from a need to recharge batteries contained within intraoral devices such as smart retainers, aligners, nightguards or oral appliance therapy (OAT) devices. The invention recognises that such devices can best be charged in a case that stores them.

The case is designed to be portable and therefore compact enough to minimise encumbrance to the user. The major dimensions such as height, width and depth of the case are optimised about the intraoral device and encapsulated electronic components of the case to ensure that the case is as small as possible with the aim of being pocket sized. The case of the invention is designed to be similar in size to existing protective cases for mouthguards to align with market expectation of size.

Ensuring proper alignment of the charging means to the device contained within the case is essential to the charging operation. The invention achieves this by mechanically constraining the intraoral device within the closed case in all degrees of freedom. The invention utilises common, repeatable geometry of at least part of the intraoral device to capture it within the case. The invention achieves this by providing retention features or formations within, around or beside the protective volume that together create a negative of the common features on the intraoral device. Additionally, or alternatively, at least one opposing pair of magnets may be used as retention features to hold the device fast within the protective volume of the case.

Part of the intraoral device, such as a retainer to engage a user's teeth, can be custom made with varying shapes and sizes depending on the user. The portion of the protective volume that houses that part of the intraoral device may be sized to account for all or most percentiles of intraoral device. In other words, the case may be sized to accommodate the largest intraoral device that is expected to be encountered, which ensures that all smaller sizes of the intraoral device will fit within the protective volume.

The abovementioned retention features ensure that the intraoral device is held fast, regardless of the volume of the casing that the device is contained within. Consequently, the case does not need be custom made, which allows a single case design to suit intraoral devices of all expected sizes. Alternatively, the case can be custom made to suit a particular intraoral device intended to be held within. Custom manufacture can optimise the fit of the intraoral device within the case and minimise the external dimensions of the case for a given size of intraoral device. Whilst custom made solutions may have advantages such as these, those advantages must be balanced against the likelihood of increased cost.

Typically, the battery and electronics of smart intraoral devices are completely encapsulated so they do not succumb to the hostile environment of the user's mouth or to the cleaning products used to sanitise the intraoral device. This complete encapsulation drives a demand for contactless charging of the battery or other energy storage device such as a capacitor. Contactless charging may be effected by an inductive charging system that provides for contactless transmission of electromagnetic energy through the use of corresponding induction loops or coils. Maintaining proximity and adequate alignment between those coils is necessary for the charging system to function.

The transmitting induction coil of the case suitably corresponds in size to the receiving coil of the intraoral device to maximise the efficiency of energy transfer. Retention formations of the case position the intraoral device such that the opposing induction coils of the intraoral device and the case are aligned and that the device is held fast when within the protective volume of the charging case. Standard common geometry across all intraoral devices for use with the protective charging case ensures simple, reliable and repeatable charging coil alignment.

Embodiments of the invention to be described receive electromagnetic energy from an external power source via a charging input such as a receiving induction loop located on the underside of the case and transfer that energy to a charging output such as a transmitting induction loop within the peripheral wall of the case from where that energy is then transmitted to the intraoral device. The elements of the inductive charging system may, for example, be compatible with the Qi wireless open interface charging standard.

Contactless charging input removes the requirement for the case to have a socket and a wired means of power transmission, which would present challenges when immersing the case for the purpose of cleaning.

By using a standard contactless charging interface, the invention fits in well with the pre-existing ecosystem of contactless chargeable devices. For example, a user may already have a charging pad or mat for charging other devices such as a mobile phone. Conveniently, therefore, the user can charge the invention with pre-existing equipment to which they may already have access.

In another embodiment, the charging system electronics for the case may be encapsulated in a charging system module that is detachable from a body of the case. This allows the electronic components of the case to be detached from the body for the purpose of cleaning the body. The body may be immersed for cleaning purposes when the charging system module is removed. Beneficially, this permits the use of a wired power transmission from an external power source, for example via a USB-C connector. However, it remains possible to charge an onboard energy store by using the wireless charging functionality of the charging system module.

Additionally, or alternatively, it is possible to provide a range of charging system modules depending on the external interface desired by the user. For example, a user may prefer a wired interface, or perhaps they wish to use a wireless charger that adheres to an alternative wireless charging standard. Thus, modularity allows flexibility in design and customisation options.

The charging system module may attach to the body of the case in different ways. For example, the charging system module and the body of the case may feature complementary rail formations allowing the module to be slid into place in an inward direction from the peripheral wall. That direction may be substantially parallel to the base of the case. Alternatively, the module may attach to the body in a direction substantially orthogonal to the base of the case. When the module is united fully with the body, moulded-in clip or detent features may prevent the module from unintentionally separating from the body, hence allowing the module to be attached securely to the body without use of tools. However, the module could instead or additionally be fastened to the body with fasteners such as screws.

It is possible that the geometry of the case may be adapted to fit all manner of intraoral devices that are compatible with inductive charging. The geometry of the case may be modified but the principles of retaining and subsequently charging the device remain the same.

The case is constructed from a suitably rigid material, for example injection moulded from a polymer such as acrylonitrile butadiene styrene (ABS), although other methods of manufacture may be used. As the top and bottom halves of the case are designed to be similar in shape and size, there is an opportunity to use a multi-core tool in which both halves can be moulded simultaneously, increasing manufacturing efficiency.

Advantageously, the material of the case is compatible with cleaning and immersion to sanitise the intraoral device and its protective case, therefore supporting the user's oral hygiene. Potentially the intraoral device can be sanitised when within the case, thus also sanitising the case at the same time.

The casing may have transparency or translucency, either fully or locally, that allows a user to view a status indicator of the intraoral device without having to open the protective case. Advantageously, this feature allows the user quickly to check the status of the intraoral device with minimal interaction or potential exposure. For example, the user can receive positive feedback that the system is charging as otherwise the user may not be warned of a failure to charge, especially when the case placed on a wireless charger.

The protective case may have its own status indicators, for example, whether the case is in a charging mode or whether there is a fault in the system. These indicators could be disposed at either the side or the top of the case for visibility while the case is in position for charging.

The case may feature an integrated energy store such as a battery or a capacitor. This brings the added benefit of charging the device within the case when away from a mains power supply. This can be particularly useful for the user during travel where the user may not have access to an external power supply such as a charging mat or may not have time to leave the case connected to one.

The inventive concept embraces a corresponding method of charging an intraoral device while it is stored in a protective volume. The method comprises aligning a charge receiver of an intraoral device with a charging output of the case. The charge receiver and the charging output may, for example, be induction coils or otherwise operate contactlessly. A charging input of the case may be coupled with an external charger such as an induction charger, allowing electromagnetic energy to flow through the case between the input and the output acting as a relay to transfer energy between the external charger and the intraoral device.

Key points:
The dental retainer is bespoke, and of variable size, for the patient but the sensing device attached to it is of a fixed size for all patients.
This enables the dental retainer with the attached sensing device to be precisely positioned in the case when the sensing device clips into the pouch part of the case with reciprocal dimensions to it.
This precise alignment enables the receiving coil in the sensing device to be aligned with the transmitting coil in the case.
The wireless recharging unit is encapsulated in its own enclosed part of the case. This enclosed part of the case is a module which may be permanently attached to the case or it may be removable. If it is removable, generally, screws will be used to attach and detach it.
The wireless recharging unit comprises a transmitting coil and its own receiving coil. This may be augmented by its own battery and supporting electrical circuit.
The wireless recharging module of the case is generally completely enclosed facilitating the cleaning of the case in the same way as hitherto.
In the situation where instead of a receiving coil, the wireless recharging unit is powered through a socket, say a USB port, introducing an opening in the wireless recharging unit, the wireless recharging unit can be removed from the case. This enables the case to be cleaned in the normal way.

### BRIEF DESCRIPTION OF THE DRAWINGS

. In order that the invention may be more readily understood, reference will now be made, by way of example, to the accompanying drawings in which:

FIG. 1 is a schematic perspective view of an embodiment of the invention comprising a charging case shown in a closed condition;

FIG. 2 is a schematic perspective view corresponding to FIG. 1 but showing the case when open and also showing an intraoral device within the case;

FIG. 3 is a cross-sectional view showing the open case with the intraoral device inserted into a lower part of the case;

FIG. 4 corresponds to FIG. 3 but shows the case closed around the intraoral device, illustrating retention features that locate the intraoral device within the case;

FIG. 5 is a schematic illustration of the electrical system of the charging case as placed on a generic wireless charger;

FIG. 6 is a schematic illustration of a variant of the arrangement shown in FIG. 5 including an on-board battery;

FIG. 7 is a schematic illustration of an alternative embodiment of the charging case showing a magnetic coupling instead of induction coils;

FIG. 8 is a pair of perspective views that show an embodiment of the case with a charging system module that is detachable from a body of the case;

FIG. 9 is a central cross-section in a plane perpendicular to the hinge axis of the case showing an embodiment having complementary rail formations between the body and the charging system module.

FIGS. 10A and 10B are schematic perspective views of an alternative embodiment of the invention showing how the intraoral device is inserted into the case;

FIG. 11 is a schematic perspective view corresponding to FIGS. 10A and 10B but showing the case when closed;

FIG. 12 is a schematic perspective view corresponding to FIG. 11 but showing the case when open;

FIG. 13 is a schematic perspective view corresponding to FIG. 12 but showing the case disassembled for cleaning;

FIG. 14 is a schematic perspective view corresponding to FIGS. 10A and 10B but with components omitted for clarity;

FIG. 15 is a schematic view on arrow A found in FIG. 14; and

FIGS. 16A, 16B, 16C and 16D show a section view corresponding to FIGS. 10A and 10B.

### DETAILED DESCRIPTION

A specific embodiment of the present invention will now be described in which various features will be discussed in detail to provide a thorough understanding of the inventive concept as defined in the claims. However, it will be apparent to the skilled person that the invention may be put into effect without the specific details and that in some instances, well known methods, techniques and structures have not been described in detail in order not to obscure the invention unnecessarily.

In overview, the invention provides a protective charging case 100 comprising an upper portion or top 10 and a lower portion or base 12, together forming a protective volume or storage compartment for an intraoral device 14 such as a smart retainer, aligner, nightguard, mouthguard, splint, or oral appliance therapy (OAT) device. A charging system of the case receives energy from an external power source to convey that energy to the intraoral device 14. Locating means are provided to hold the intraoral device 14 in place to ensure that the charging process is not interrupted by undesired displacement of the intraoral device 14 within the protective volume.

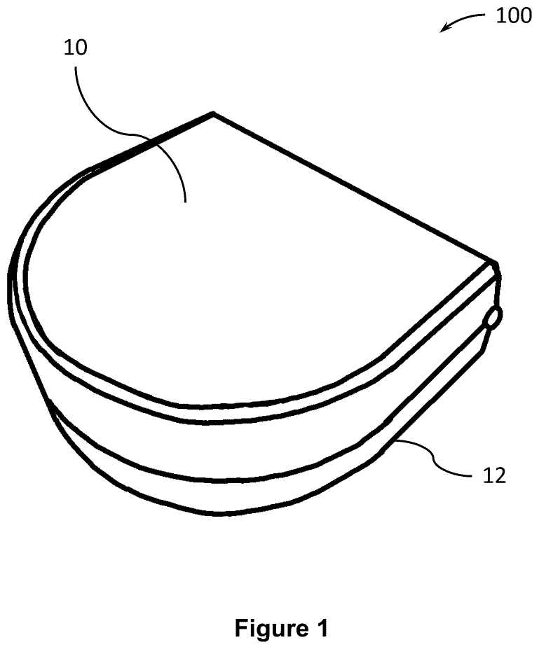

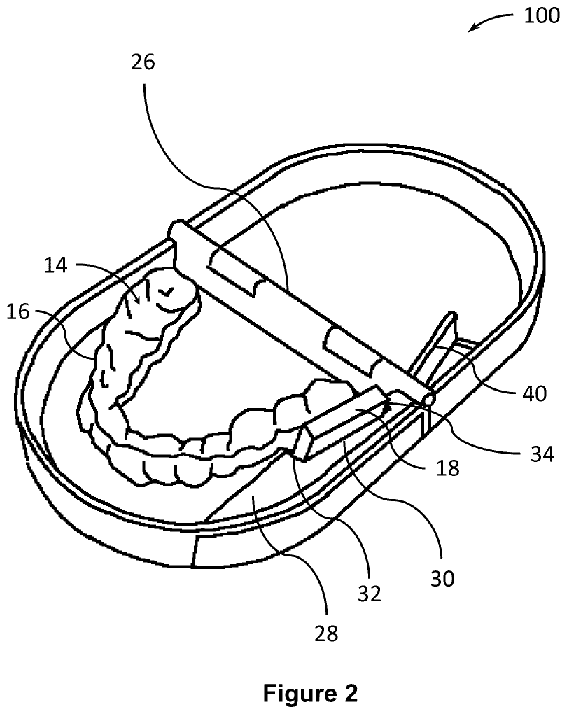

In this example, as can be seen when the case shown closed in FIG. 1 is opened as shown in FIG. 2, the intraoral device 14 comprises bespoke portion 16, for example a U-shaped mouthguard or nightguard, supporting a sensor module 18 at the end of a limb of the U-shape. The sensor module 18 may, for example, be capable of detecting masseter muscle activity that characterises bruxism. The sensor module 18 comprises a shallow generally cuboidal housing that protrudes laterally from the curvature of the nightguard, presenting one of the major faces of the housing in an outward lateral direction. The geometry of the sensor module 18 is intended to be common between various such intraoral devices 14.

The dimensions and shape of the case 100 fit closely around an intraoral device 14 intended to be contained in the protective volume and around the electronic components of the case that facilitate the charging process. Specifically, the external contours of the case 100 and of the protective volume within are defined by a top wall 20, a bottom wall 22 and a peripheral wall 24 that joins the top and bottom walls 20, 22. The peripheral wall 24 has two sections, one section being curved in a U shape and the other section being substantially flat or straight between the limbs of the U shape. The curved section may be considered as the front of the case and the flat section may be considered as the rear of the case.

The case 100 is divided through the peripheral wall 24 in a plane parallel to the bottom wall 22 forming two halves or portions that are articulated relative to one another about a hinge 26 disposed to the rear of the case. Opening the upper portion about the hinge 26 opens the protective volume to allow insertion or removal of an intraoral device 14 to be charged within the case 100.

Location features disposed beside, around or within the protective volume engage with the intraoral device 14 to hold the device in place, ensuring that there is minimal relative movement between the intraoral device 14 and the case 100 and hence maintaining alignment of the charging system. These features will now be described in detail.

In the lower portion 12 of the case 100, there is a protrusion 28 that encapsulates the charging system of the case. The protrusion is defined by a local thickening or widening of the peripheral wall 24, in this example being offset to one side of the case on one limb of the U-shaped peripheral wall 24. This defines a chamber that contains most or all of the electronics of the charging system.

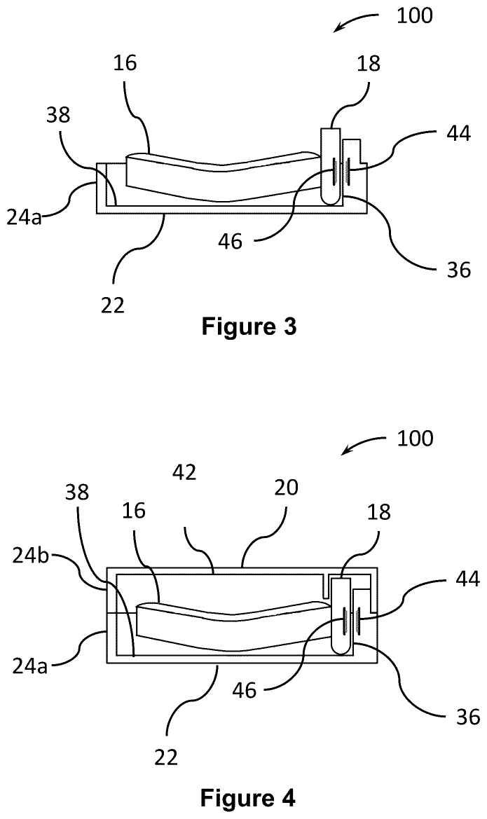

Referring now also to FIGS. 3 and 4 of the drawings, an inwardly-facing female formation in the form of a recess, slot or socket 30 located at or close to the extremity of a limb of the U-shaped peripheral wall 24 is sized to receive the sensor module 18 of the intraoral device 14. In this embodiment, the inward direction is inward with respect to the peripheral wall 24, in a direction extending from one side of the curved section of the wall 24 to the other side of that curved section. The recess 30 has a forward wall 32, a rearward wall 34 and a laterally outward wall 36 and defines an upwardly-open pocket that complements and receives a lower portion of the sensor module 18 of the intraoral device 14. The floor 38 of the protective volume constrains the intraoral device 14 in the downward vertical direction. In this example, the recess is formed in the protrusion 28, thus being a location where the peripheral wall 24 is not as thick or wide as the remainder of the protrusion 28.

The laterally outward wall 36 of the recess 30 need not be parallel to the peripheral wall 24 of the case 100 that is laterally outward wall of the recess 30. In this example, the laterally outward wall 36 of the recess 30 diverges from the laterally outward wall of the case 100 in a direction away from the planar section of the peripheral wall 24. This feature ensures that the case 100 is capable of accommodating any practical embodiment of the intraoral device 14.

Moving now to the upper portion 10 of the case 100, the upper portion 10 also has a feature that engages the sensor module 18 of the intraoral device 14. This feature takes the form of a wall 40 that, when the case 100 is closed, is opposed parallel to and spaced inwardly from the recess 30 in the lower portion 12 of the case 100. This defines a downwardly-open pocket between the wall of the upper portion 10 and the outer wall 36 of the recess 30, which pocket receives an upper portion of the sensor module 18 of the intraoral device 14. The wall 40 thereby captures the sensor module 18 of the intraoral device 14 upon closing the case 100 and provides constraint in an inward lateral direction. The upper inner surface 42 of the upper portion 10 constrains the intraoral device 14 in the upward vertical direction.

Together, the wall 40 and recess 30 form a negative of the geometry of the sensor module 18 of the intraoral device 14, thus embracing the sensor module 18 between them and capturing the intraoral device 14 when it is introduced into the protective volume and the case 100 is closed. The combination of these features adequately constrains the intraoral device 14 in all degrees of freedom.

Elegantly, the asymmetric retention geometry described above makes insertion of the intraoral device 14 into the protective case 100 a poka-yoke process. In other words, the intraoral device 14 can only be engaged with the case 100 in one orientation. It is not possible for the user to close the case 100 if the intraoral device 14 has not been inserted correctly.

As the intraoral device 14 can only be installed in the case in one way, and the act of closing the case 100 ensures that the intraoral device 14 is prevented from moving, the charging elements responsible for transferring energy from the case 100 to the intraoral device 14 are securely aligned and sufficiently close to one another to allow for repeatable and efficient charging.

Another embodiment of the invention may additionally or alternatively employ one or more pairs of opposing location magnets that can be positioned in the case 100 and the intraoral device 14 to ensure that alignment of the charging elements is maintained. This approach could also help to retain the intraoral device 14 within the case 100 before closing the case 100.

The charging elements to transfer energy from the case 100 to the intraoral device 14 may comprise a complementary pair of induction coils. This approach allows for contactless transmission of electromagnetic energy. The functional detail of transferring energy by means of induction coils will be well known to those skilled in the art and therefore will not be discussed in this document.

In order for this contactless method of charging to function, the complementary coils on or encapsulated within the intraoral device 14 and the case 100 must be substantially aligned axially and proximate to one another. Thus, the transmission coil 44 in the case serving as a charging output is located on or in the peripheral wall 24 in the lower portion 12 of the case 100 in the recess 30. This location is closely proximate to the position of the receiving coil 46 in the sensor module 18 of the intraoral device 14, when the sensor module 18 is received in the recess 30. Although some deviation can be tolerated, the function and efficiency of the charging system requires that any misalignment or displacement is kept to a minimum. This reinforces the benefit of the retention geometry mentioned above.

Electronics of the charging system are located in the internal protrusion 28 of the lower portion 12 of the case 100. Locating them in this region of the case ensures that they are consolidated and thus the design of the moulding to contain them is simplified. The electronics are encapsulated to minimise risks of fluid ingress when cleaning the apparatus. As such, the same challenges regarding charging apply to the case 100 as they do to the sensor module 18 of the intraoral device 14.

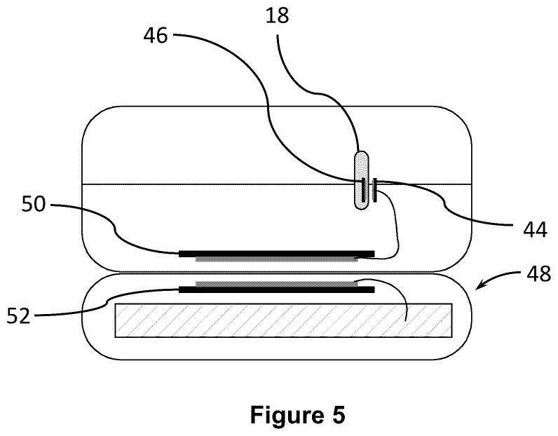

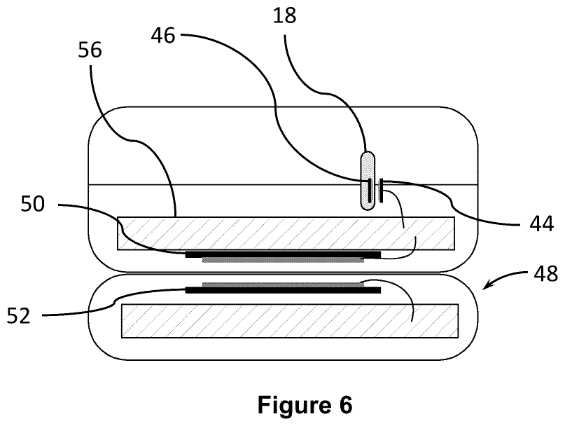

The case 100 receives electrical charge from an external power source 48. In the embodiment described above, the electronics of the case are encapsulated, driving a requirement for contactless energy transfer. Consequently, in the examples described, input of energy to the case is also effected via a complementary pair of induction coils. Reference is made to FIGS. 5 and 6 of the drawings in this respect.

The receiving induction coil 50 constituting a charging input of the case 100 is located in or on the substantially flat bottom surface 22 of the case 100. Again, that case may conform to an industry standard for wireless charging, for example, being compatible with the Qi wireless open interface charging standard. In that way, the case will be compatible with an existing ecosystem of wireless chargers. The substantial flatness of the underside of the case allows for stable interaction with a standard charging mat or pad and for the receiving coil 50 to be aligned and proximate to the transmission coil of the charger 52.

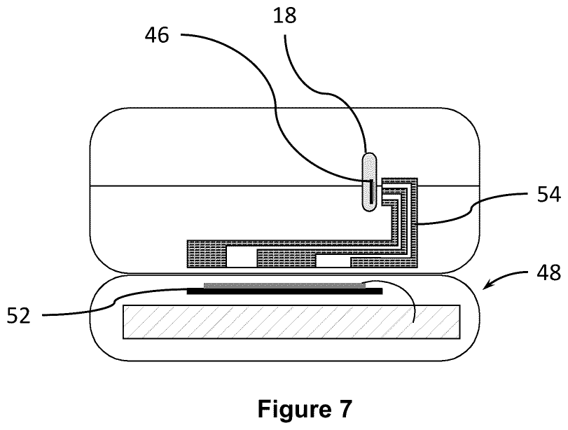

In another embodiment, it is possible for the electrical charge to be transferred via a magnetically conductive medium 54 such as ferrite or iron alloy instead of induction coils. This is shown schematically in FIG. 7.

It is possible that the case 100 could receive electrical energy from a wired interface, for example via a USB-C connector. However, additional means of preventing fluid ingress may then be necessary to ensure that the case does not succumb to exposure to environments that could be harmful to electronic devices, for example when immersed in water or other cleaning fluid.

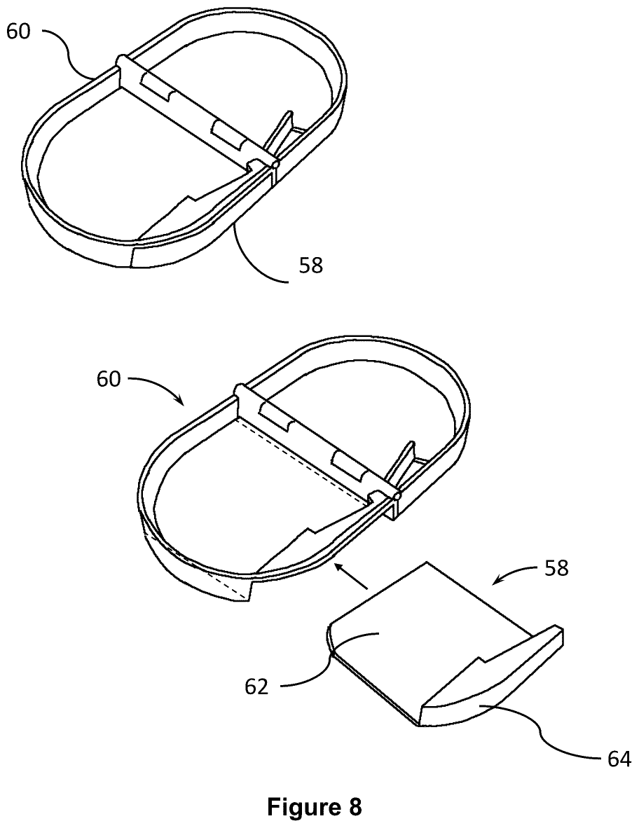

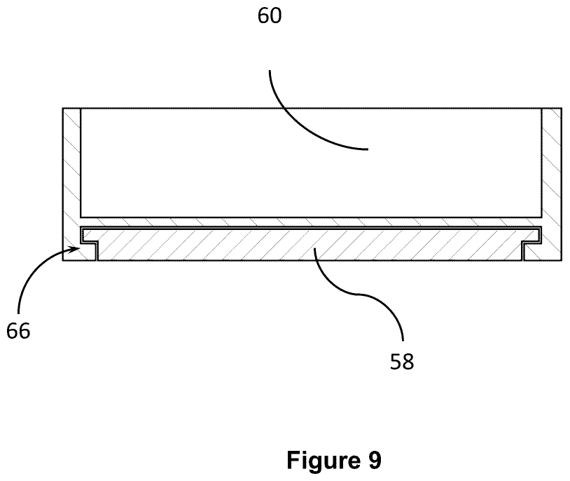

Turning finally to FIGS. 8 and 9, these drawings exemplify how charging system electronics may be encapsulated in a charging system module 58 that is detachable from a body 60 of the case. Beneficially, the body 60 is less sensitive to repeated immersion in a harsh cleaning environment than the charging system module 58. The general layout of the electronics, in particular the location of the receiving and transmission coils with respect to the case, is similar to that described above.

The charging system module 58 may be substantially L-shaped in side view, with an elongate base 62 containing the receiving coil 50 and a comparatively short upright 64 containing the transmission coil 44 lying in a plane substantially orthogonal to the plane of the bottom wall 22. The body 60 of the case may feature a complementary recess to accept the module 58.

In this example, the charging system module 58 attaches to the body 60 of the case in an inward direction from the peripheral wall of the case and forms a substantial portion of the bottom and peripheral walls of the case. The module 58 is attached via complementary rail formations 66 moulded into both the body 60 and the module 58, as best shown schematically in FIG. 9. The rail formations 66 extend inwardly from the peripheral wall in a direction substantially parallel to the bottom wall 22 of the case. When pushed completely into the body, moulded-in clip or detent features may prevent the module 58 from unintentionally separating from the body 60 if friction alone is deemed insufficient.

Other methods of attachment are possible in addition or alternatively. For example, screws or other fasteners could be used to hold the charging system module 58 to the body 60. It is also possible that the module 58 could be attached to the body 60 without the use of the abovementioned complementary rail formations 66. Instead, for example, the module 58 could be offered up to the body 60 in a direction that is orthogonal to the bottom wall 22 of the case 100. The module 58 may then be held in place by push-fit pins received in holes where the pins exist in the module 58 and the holes exist in the body 60 or vice versa. In this example, screws could also be used to hold the module 58 to the body 60.

Many other variations are possible within the inventive concept. For example, the transmission coil of the charging system may be disposed in a protrusion of the charging system module 58. In that instance, an aperture in the laterally outward wall 36 of the recess 30 in the body 60 could receive the protrusion on the module 58. In this way, the distance and thickness of material between the transmission coil 44 of the charging system and the receiving coil 46 of the device to be charged is minimised when the module 58 is assembled to the body 60.

In the embodiment shown in FIG. 6, the invention has a means of storing electrical charge on board. Typically, this will be by way of a rechargeable battery 56, but other energy storage means such as capacitors are possible. With on-board energy storage, the case 100 may be charged in isolation without the intraoral device 14 necessarily being present. This stored electrical charge can then be used at a later stage to charge the intraoral device 14 when required.

As there is significantly more space to house a battery 56 in the protective case 100 when compared to the intraoral device 14, there is an opportunity to include a battery 56 in the case 100 with a much greater charge capacity then the internal battery of the intraoral device 14. This would allow a user to charge the intraoral device 14 multiple times from just one charge of the battery 56 within the case.

The case 100 may be transparent or translucent, either fully or locally, to allow visibility of a status indicator of the intraoral device 14. For example, the user may be able to ascertain from that indicator whether the intraoral device 14 is in a charged state or if it is actively being charged.

In another embodiment, the case 100 may have its own status indicators. From those indicators, the user may be able to ascertain any or all of the following:
the charge state of the case;
the charge state of the intraoral device within the case;
whether an intraoral device is present within the case;
if the case is being charged;
if the intraoral device is being charged;
a low battery warning for the case;
a low battery warning for the intraoral device; and/or
a fault warning for the charging system in general.

In another embodiment of the invention, a protective charging case 200 comprises an upper portion or top 210 and a lower portion or base 212, together forming a protective volume or storage compartment for an intraoral device 14 such as a smart retainer, aligner, nightguard, mouthguard, splint, or OAT device. A charging system of the case 200 receives energy from an external power source and conveys that energy to the intraoral device 14. Locating formations are provided to hold the intraoral device 14 in place to ensure that the charging process is not interrupted by undesired displacement of the intraoral device 14 within the protective volume.

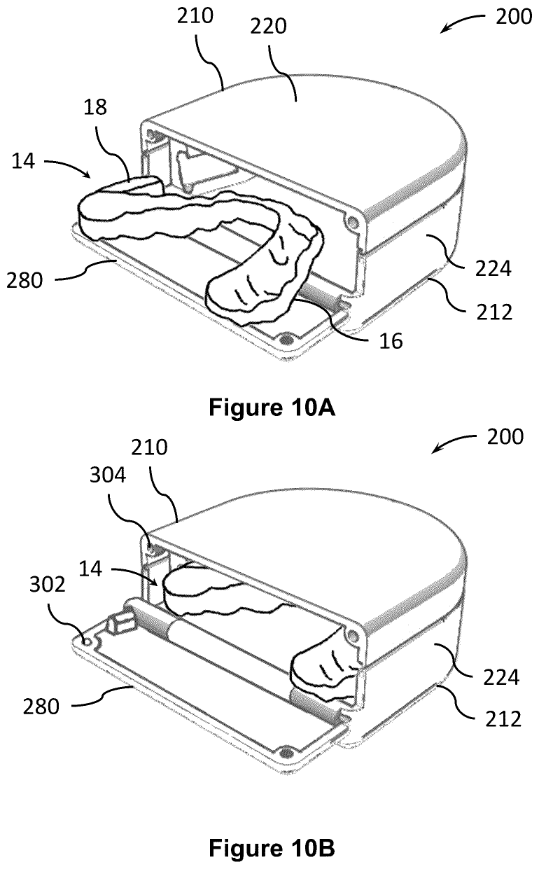

In this example, as can be seen when the case 200 shown closed in FIGS. 10A and 10B, the intraoral device 14 comprises a U-shaped mouthguard or nightguard supporting a sensor module 18 positioned externally at the end of a limb of the U-shaped bespoke portion 16. The sensor module 18 comprises a shallow generally cuboidal housing that protrudes laterally from the curvature of the bespoke portion 16, presenting one of the major faces of the housing in a lateral direction. The geometry of the sensor module 18 is intended to be common between various such intraoral devices 14. In other words, the bespoke U shaped portion 16 of the intraoral device 14 may vary from one device to another but the sensor module 18 is a standardised component with common geometry as stated above.

As before, the dimensions and shape of the case 200 fit closely around the intraoral device 14 intended to be contained in the protective volume and around the electronic components of the case that facilitate the charging process. Specifically, the external contours of the case 200 and of the protective volume within are defined by a top wall, a bottom wall and a peripheral wall 224 that joins the top and bottom walls. The peripheral wall 224 has two sections, one section being curved in a U shape and the other section being substantially flat or straight between the limbs of the U shape.

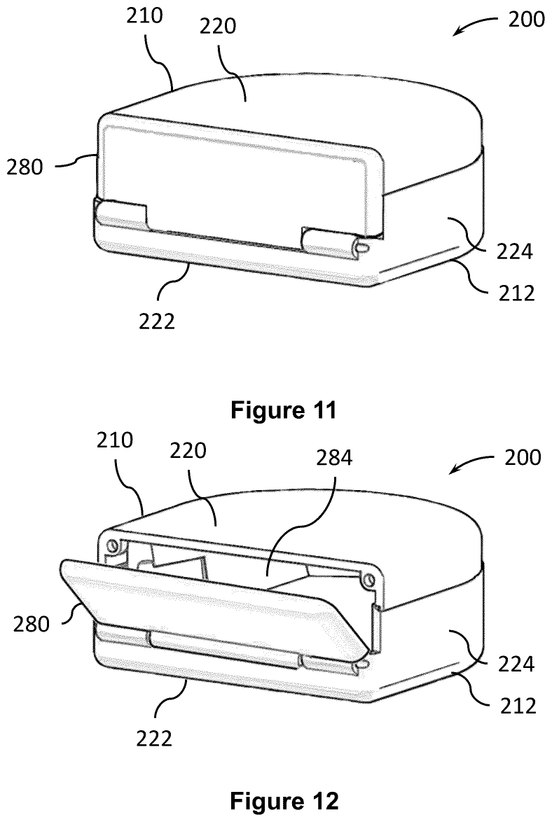

As can be seen in FIGS. 11 and 12, the interior of the case 200 is accessed via a hatch or a door 280 disposed on the substantially flat or planar portion of the peripheral wall 224 extending between the limbs of the U shape. A hinge 282 is positioned on this portion of the peripheral wall 224 to allow the door 280 or hatch to open by articulating about the axis of the hinge. The hinge axis is located on an external face of the planar wall, in a position offset towards and substantially parallel to the bottom wall 222 of the case 200. Opening the hatch or door 280 about the hinge 280 opens the protective volume to allow insertion or removal of an intraoral device 14 to be charged within the case 200. Specifically, opening the hatch or door 280 opens an aperture 284 in the case 200 through which the intraoral device 14 can pass during insertion and removal.

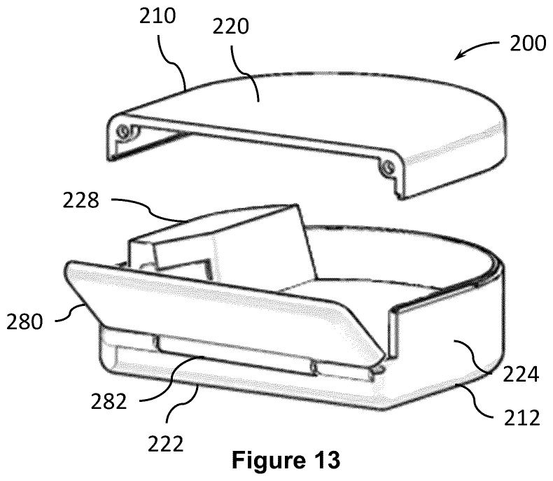

In this example, the case 200 is divided through the peripheral wall 224 in a plane parallel to the bottom wall to form two separate portions, a top portion 210 and a bottom portion 212. Here, the two portions (210, 212) of the case 200 are separable as best appreciated in FIG. 13. However, the portions (210, 212) could instead be articulated relative to one another about a hinge (not shown) disposed centrally on the curved portion of the peripheral wall 224 of the case 200. Separating or moving apart the portions (210, 212) of the case 200 provides unrestricted access for a user to sanitise the interior surfaces of the case 200.

Location features disposed beside, around or within the protective volume engage with the intraoral device 14 to hold the device in place, ensuring that there is minimal relative movement between the intraoral device 14 and the case 200 and hence maintaining alignment necessary for the charging system to operate. These features, which act on the sensor module 18 of the intraoral device 14, will now be described in detail.

In the lower portion 212 of the case 200, there is a protrusion 228 that encapsulates the charging system. The protrusion 228 is defined by a local thickening or widening of the peripheral wall 224, in this example being offset to one side of the case 200 on one limb of the U-shaped peripheral wall 224. The protrusion 228 is hollow to define a chamber that contains some or all of the electronics of the charging system.

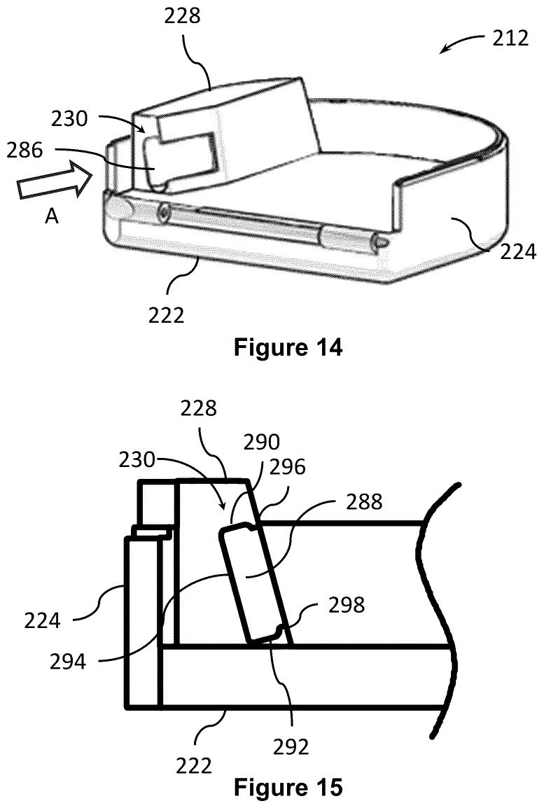

Referring now also to FIG. 14 and FIG. 15 of the drawings, a female formation in the form of a recess, slot or socket 230 is located at or close to the extremity of a limb of the U-shaped peripheral wall 224. The socket 230 is sized and shaped to receive and to complement the size and shape of the sensor module 18 of the intraoral device 14. Formations defining the socket 230 are elongated in a direction of insertion/removal of the intraoral device 14 into and out of the case 200 through the aperture 284. Also, the socket 230 is open at its end facing toward the aperture 284 and closed at its end remote from the aperture 284.

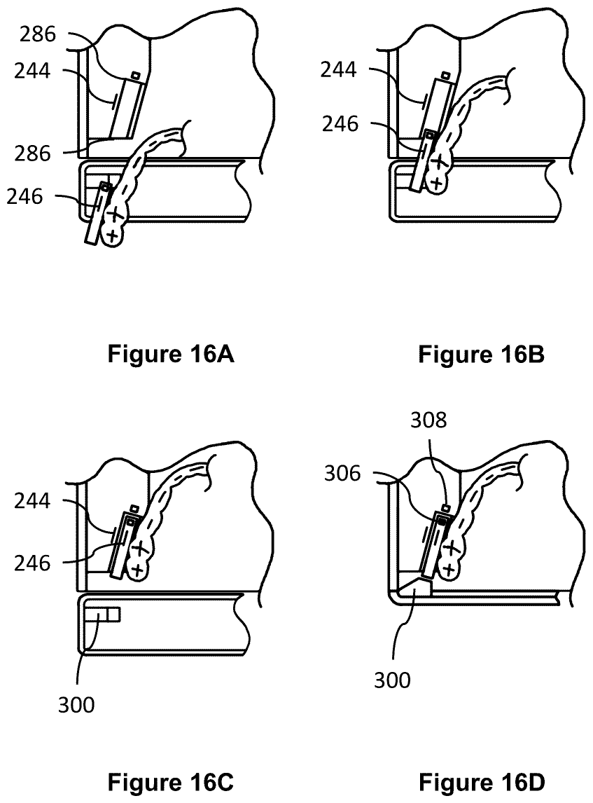

Referring now to the step-by-step process depicted in FIGS. 16A to 16D, upon insertion of the intraoral device 14 to the protective volume through the aperture 284 in the peripheral wall 224 of the case 200, the sensor module 18 enters the socket 230 through the open end 286. The sensor module 18 then slides along the socket 230 upon further insertion into the case 200. Eventually, the closed end 288 of the socket 230 serves as a stop formation that blocks inward movement of the sensor module 18 of the intraoral device 14 when the inward end of the sensor module 18 encounters the closed end 288 of the socket 230. When the sensor module 18 is in that position, the charging coils (244, 246) of the case 200 and the intraoral device 14 are in mutual alignment.

The direction of insertion need not necessarily be orthogonal to the plane of the aperture 284; indeed, in this example, the direction of insertion is at an acute or obtuse angle to the plane of the aperture 284. This feature ensures that the case 200 is capable of accommodating any practical embodiment of the intraoral device 14.

Furthermore, although the peripheral wall 224 is perpendicular to the base of the case 200, the recess geometry may not necessarily follow suit. Instead, the recess geometry may form an acute or obtuse angle to the base of the case 200. Similar to above, this feature ensures that the case 200 is capable of accommodating any practical embodiment of the intraoral device 14.

The socket 230 forms a negative of the geometry of the sensor module 18 of the intraoral device 14, thus embracing the sensor module 18 and capturing the intraoral device 14 when it is introduced into the protective volume and the case 200 is closed. Movement in upward and downward directions is restricted by upper and lower walls (290, 292) of the socket 230, each extending in the direction of insertion of the intraoral device 14 and substantially parallel to each other.

Lateral movement of the sensor module 18, and hence of the intraoral device 14, is restricted by a laterally outward wall 294 of the socket 230. The socket 230 also has a laterally inward side that comprises a pair of flanges (296, 298) protruding from the upper and lower walls (290, 292) and extending in the direction of insertion of the intraoral device 14. An elongate gap or slot between the flanges (296, 298) provides clearance, and therefore allows passage, for the bespoke U shaped portion 16 of the intraoral device 14. These flanges (296, 298) engage the sensor module 18 to restrict laterally inward movement of the intraoral device 14 relative to the case 200. The combination of these formations of the socket 230 substantially constrains the common geometry of the sensor module 18, and therefore the entirety, of the intraoral device 14 in all degrees of freedom within the case 200.

It can therefore be appreciated that the protective volume of the case 200 comprises a volume subdivided into two parts: the socket 230 for closely fitting around the sensor body of the intraoral device 14 with the purpose of retaining the intraoral device 14; and the storage compartment for protecting the bespoke geometry 16 of the intraoral device 14 when the device is located within the confines of the case 200. These two volume portions are joined via the abovementioned slot that permits the intraoral device 14 to occupy both portions at once when inserted into the case 200. The storage compartment is anthropometrically sized to accommodate a majority of sizes and shapes of the bespoke geometry 16 of an intraoral device 14, a geometry that is primarily based on at least a part of a user's dentition.

As is also appreciated in FIGS. 16A to 16D, the open end 286 of the socket 230 is positioned close enough to the aperture 284 as, effectively, to be closed by the door 280 when the door 280 itself is closed. In other words, closing the door 280 prevents the sensor module 18 from disengaging from the socket 230, by being in close proximity to, touching or bearing on either the U shaped portion of the intraoral device 14 or upon the sensor module 18 itself. The door 280 features an inwardly projecting formation 300 which is in alignment with the open end 286 of the socket 230 when the door 280 is closed. This formation 300 is present to bridge any gap between the door 280 and the sensor module 18 of the intraoral device 14 when it is positioned in the case 200.

As with the preceding embodiment, the asymmetric retention geometry described above makes insertion of the intraoral device 14 into the protective case 200 a poka-yoke process. Similarly, it is not possible for the user to close the case 200 if the intraoral device 14 has not been inserted correctly.

As the intraoral device 14 can only be installed in the case 200 in one way, and the act of closing the case 200 ensures that the intraoral device 14 is prevented from moving, the charging elements responsible for transferring energy from the case 200 to the intraoral device 14 are securely aligned and sufficiently close to one another to allow for repeatable and efficient charging.

The hatch or door 280 is fastened in a closed position preferably by mutually opposed magnets (302, 304) in both the hatch or door 280 and the body of the case 200. This means of fastening is not intended to be limiting as a mechanical latch or detent could be used as an alternative, for example.

Similarly, the two portions (210, 212) of the case 200 may be fastened together magnetically or mechanically.

Furthermore, the inward end of the sensor module 18 on the intraoral device 14 may comprise a magnet 306 that is mutually opposed to another magnet 308 positioned at the closed end of the socket 230 in the case 200. This pair of mutually opposed magnets (306, 308) not only aids in the retention of the intraoral device 14 in the case 200 but helps to ensure that the respective charging coils (244, 246) of the intraoral device 14 and the case 200 are brought into and kept in alignment with one another. Of course, it would instead be possible to place a magnet in only one of the parts, that magnet being attracted to a compatible metallic insert in the other part.

In all embodiments of the invention, the contactless charging output coil of the case 200 may comprise electrical contacts instead.

1. A charging case for an intraoral device, the case comprising:
a storage compartment surrounded by a peripheral wall extending between a top and a base; and
a charging output disposed within a recess in the peripheral wall and facing into the storage compartment.

2-57. (canceled)
